(19) United States
(12) Patent Application Publication
(10) Pub. No.: US 2021/0257067 A1
(43) Pub. Date: Aug. 19, 2021

(54) STATE TRANSITION PREDICTION DEVICE, AND DEVICE, METHOD, AND PROGRAM FOR LEARNING PREDICTIVE MODEL

(71) Applicant: NIPPON TELEGRAPH AND TELEPHONE CORPORATION, Tokyo (JP)

(72) Inventors: Tsutomu YABUUCHI, Musashino-shi, Tokyo (JP); Shozo AZUMA, Musashino-shi, Tokyo (JP); Naoki ASANOMA, Musashino-shi, Tokyo (JP); Akihiro CHIBA, Musashino-shi, Tokyo (JP); Kana EGUCHI, Musashino-shi, Tokyo (JP); Tomohiro YAMADA, Musashino-shi, Tokyo (JP); Hisashi KURASAWA, Musashino-shi, Tokyo (JP); Kazuhiro YOSHIDA, Musashino-shi, Tokyo (JP)

(73) Assignee: NIPPON TELEGRAPH AND TELEPHONE CORPORATION, Tokyo (JP)

(21) Appl. No.: 17/271,177

(22) PCT Filed: Aug. 22, 2019
(86) PCT No.: PCT/JP2019/032900

§ 371 Date: Feb. 24, 2021

(30) Foreign Application Priority Data
Aug. 31, 2018 (JP) 2018-163515

(51) Int. Cl. G16H 10/60; G06N 5/04; G06N 20/00

(52) U.S. Cl. CPC G16H 10/60; G06N 20/00; G06N 5/04

(57) ABSTRACT

According to one embodiment of the present invention, sets of medical record data that have a common development order of diseases to be focused on and different times until the diseases develop are selected from medical record data, a feature indicating a health state of a user is extracted from each piece of medical record data constituting the set for each of the sets of the medical record data, the extracted feature is set as training data, a risk score for a co-occurrence or an occurrence of a complication of each of the diseases is calculated based on examination data of a first-year examination and a time until each of the diseases occur, and the risk score is set as correct answer data. At this time, the development risk score is calculated such that a user having a short elapsed time until development has a larger value than a user having a long elapsed time until development. Then, a prediction model is generated by inputting the training data to a learning machine and causing the learning machine to learn such that the output becomes the correct answer data.

### TECHNICAL FIELD

The present invention relates to a state transition prediction device and a device, a method, and a program for learning a prediction model, for example, that are used for predicting a future disease development risk based on a user's current health state in the field of medical health.

### BACKGROUND ART

As one method for calculating a future disease development risk score based on information representing a health state of an individual, a technique for creating a score function targeted for a single disease and applying the score function has been proposed. For example, in the field of metabolic systems, diabetes and hypertension are differentiated, and a development risk score is calculated for each of the diseases (for example, see Non Patent Literature 1).

In the design of a function for calculating the development risk score, in a case that the direction of a future state transition is a single direction, periods until transitions can be compared from the viewpoint of whether a period is short or long at one axis, and selection of a model of the function and parameters need to be configured such that the evaluation of the periods at one axis becomes correct. In other words, a development/progress risk function for each disease is created in accordance with a period until a transition of the disease occurs.

### CITATION LIST

### Non Patent Literature

Non Patent Literature 1: Nanri A, et al. “Development of Risk Score for Predicting 3-Year Incidence of Type 2 Diabetes: Japan Epidemiology Collaboration on Occupational Health Study.”, PLoS One. 2015 Nov. 11; 10(11): e0142779. doi: 10.1371/journal.pone.0142779.eCollection 2015.

### SUMMARY OF THE INVENTION

### Technical Problem

Meanwhile, life style-related diseases are a group of diseases of which development and progress are greatly influenced by life styles such as dietary life, exercise habits, sleep, alcohol intake, and the like, and diabetes, hypertension, neoplasm, and the like are included therein. Lifestyle diseases are known to co-occur. For example, it is known that the likelihood of occurrence of hypertension is high for patients with diabetes. It is also known that complications from diabetes that is one of lifestyle-related diseases are diverse and include nephropathy, retinopathy, neuropathy, and the like.

However, as described in Non Patent Literature 1, in a technology of creating a score function for each disease and calculating a score of the development risk of a disease, a development/progress risk function is created in accordance with a period until a transition of one disease occurs, and thus a risk score cannot uniformly be calculated for co-occurring or combined diseases. For example, although complications developing in patients with diabetes are diverse and include nephropathy, retinopathy, neuropathy, and the like, it is difficult to calculate a risk score that can be used for comparing the degrees of progress of diabetes between patients with nephropathy and patients with retinopathy.

The present invention is in view of the situations described above and provides a technology capable of calculating a score representing the magnitude of a trend in which state transitions occur as a uniform value regardless of a pattern of the state transitions even in a case that there are a plurality of patterns of future state transitions.

### Means for Solving the Problem

In order to solve the problems described above, a state transition prediction device and a state transition prediction method according to a first aspect of the present invention include: a feature data acquiring unit configured to acquire feature data including a feature relating to a first state, an elapsed time until the first state transitions to a second state, and an elapsed time until the first state transitions to a third state in a case that a health state of a user transitions from the first state to the second state due to an occurrence of a first symptom and transitions from the second state to the third state due to an occurrence of a second symptom; a selection unit configured to select, from the acquired feature data, first feature data and second feature data, in which the first symptom of the first feature data is identical to the first symptom of the second feature data, the second symptom of the first feature data is identical to the second symptom of the second feature data, and elapsed times of state transitions are different from each other; and a prediction model generating unit configured to generate a prediction model by setting the feature relating to the first state included in each of the first feature data and the second feature data as training data and causing a learning machine to learn prediction scores which are respectively calculated based on the features and reflect the elapsed times respectively included in the first feature data and the second feature data as correct answer data.

### Effects of the Invention

According to a first aspect of the present invention, in a case that a health state of a user transitions from the first state to the second state due to an occurrence of a first symptom and transitions from the second state to the third state due to an occurrence of a second symptom, a prediction model is generated with patterns of the state transitions and elapsed times until the state transitions occur taken into account. Therefore, even in a case that there are a plurality of patterns of future state transitions, a prediction model capable of calculating the magnitude of a trend in which state transitions occur as a uniform score regardless of patterns of the state transitions can be generated.

### BRIEF DESCRIPTION OF DRAWINGS

FIG. 1 is a block diagram illustrating the functional configuration of a state transition prediction device according to an embodiment of the present invention.

FIG. 2 is a flowchart illustrating a processing sequence and processing details of a learning phase using the state transition prediction device illustrated in FIG. 1.

FIG. 3 is a flowchart illustrating a processing sequence and processing details of a prediction phase using the state transition prediction device illustrated in FIG. 1.

FIG. 4 is a diagram illustrating an example of medical record data.

FIG. 5 is a diagram illustrating an example of a period until development is reached and correct answer data for each user.

FIG. 6 is a diagram illustrating an example of a prediction model learning process in the learning phase illustrated in FIG. 2.

FIG. 7 is a diagram illustrating an example of a state transition prediction process in the prediction phase illustrated in FIG. 3.

### DESCRIPTION OF EMBODIMENTS

Hereinafter, an embodiment of the present invention will be described with reference to the drawings.

### Embodiment

In one embodiment of the present invention, a case in which development risks of co-occurrence or complications of a plurality of diseases in the future are predicted based on examination data representing a current health state of a user in the field of medical health will be described as an example.

### Configuration Example

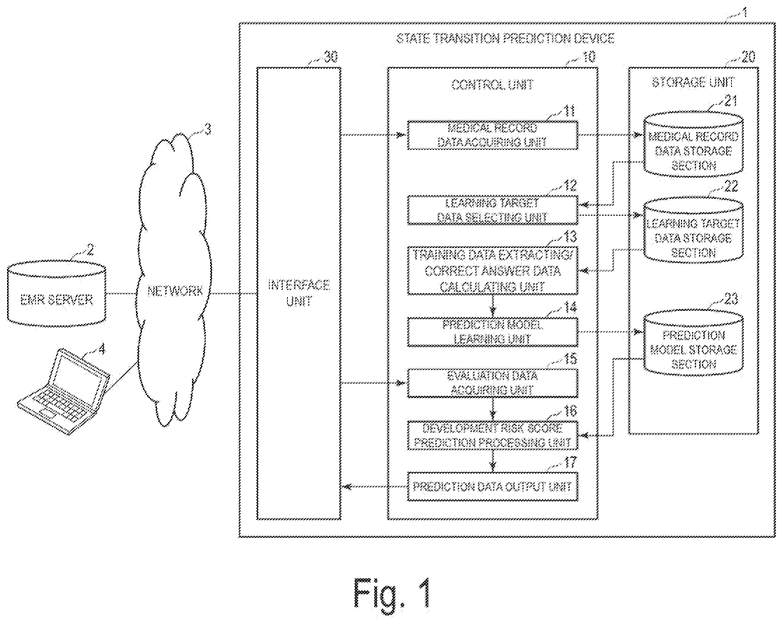

FIG. 1 is a block diagram illustrating the functional configuration of a state transition prediction device according to an embodiment of the present invention.

The state transition prediction device 1 is, for example, configured by a server computer or a personal computer and is able to communicate with an electronic medical records (EMR) server 2 and an access terminal 4 through a network 3.

The EMR server 2, for example, is located in an individual medical institution such as a hospital, a medical office, or the like and accumulates and manages medical record data including medical treatment data, examination data, inquiry data, and the like for individual patients. The EMR server 2 may be replaced with an electronic health records (EHR) server configured so as to be shared by a plurality of medical institutions within a region or a user terminal storing personal health records (PHR) data.

The access terminal 4 is, for example, a terminal used by a medical healthcare related person such as a doctor, a nurse, a public health nurse, or the like, a terminal used by a third-party receiving permission from a user such as an insurance company, or a terminal used by a user and, for example, is configured by a personal computer, a tablet-type terminal, or a smartphone.

The network 3, for example, includes a public network such as the Internet and an access network for accessing the public network. As the access network, for example, a local area network (LAN) or a wireless LAN inside the facility is used, and instead of such a network, a wired telephone network, a cable television (CATV) network, a mobile telephone network, a public wireless LAN, or the like may be also used.

The state transition prediction device 1 is, for example, located in a medical institution and is, for example, configured by a server computer. The state transition prediction device 1 may be installed alone or may be provided in a doctor's terminal, an EMR server, an EHR server, or a cloud server as one of expanded functions thereof.

The state transition prediction device 1 is realized by hardware and software. The hardware includes a control unit 10 to which a storage unit 20 and an interface unit 30 are connected through a bus that is not illustrated in the drawing.

The interface unit 30 performs data transmission between the interface unit 30 and the EMR server 2 and between the interface unit 30 and the access terminal 4 through the network 3. The interface unit 30 may also have a function of performing data transmission between the interface unit 30 and a management terminal connected through a LAN or a signal cable.

The storage unit 20, for example, is configured by combining a non-volatile memory such as a hard disk drive (HDD) or a solid state drive (SSD) that allows occasional writing and reading, a non-volatile memory such as a read only memory (ROM), and a volatile memory such as a random access memory (RAM) as storage media. A program storage region and a data storage region are provided in a storage area thereof. In the program storage region, programs that are required for executing various control processes according to an embodiment of the invention are stored.

In the data storage region, a medical record data storage section 21, a learning target data storage section 22, and a prediction model storage section 23 are configured. The medical record data storage section 21 is used for storing medical record data of a plurality of users acquired from the EMR server 2 and the like. The learning target data storage section 22 is used for storing data of a learning target selected from medical record data of a plurality of users stored in the medical record data storage section 21 described above. The prediction model storage section 23 is used for storing a learned prediction model.

The control unit 10, for example, includes a hardware processor such as a central processing unit (CPU) and, as control function units for realizing an embodiment of the present invention, includes: a medical record data acquiring unit 11; a learning target data selecting unit 12; a training data extracting/correct answer data calculating unit 13; a prediction model learning unit 14; an evaluation data acquiring unit 15; a development risk score prediction processing unit 16; and a prediction data output unit 17. All such control function units are implemented by causing the hardware processor described above to execute a program stored in the program storage region described above.

The medical record data acquiring unit 11 acquires medical record data of a plurality of users from the EMR server 2 described above through the network 3 and the interface unit 30 in a learning phase. In addition, the medical record data acquiring unit 11 performs the process of storing the medical record data in the medical record data storage section 21 in association with individual identification information of the user (a user ID) described above.

The learning target data selecting unit 12 performs the process of selecting learning target data while focusing on a plurality of diseases having a likelihood of occurring as a co-occurrence or a complication, for example, diabetes and hypertension. The kinds of the plurality of diseases to be focused on described above are not limited to diabetes and hypertension but may be other diseases such as nephropathy and retinopathy. The kinds of diseases that are the learning targets described above, for example, are designated in advance by an operation manager of the state transition prediction device 1.

The learning target data selecting unit 12 first selects, among medical record data of a plurality of users stored in the medical record data storage section 21, medical record data in which a development history of the plurality of diseases that are focused on described above is present or medical record data under tracking and observation of development of each of the diseases. Then, a plurality of sets of medical record data are selected, the set of medical record data having a common development order of the above-described diseases to be focused on, and different elapsed times until each of the diseases occurs, and each of the sets of the medical record data that have been selected is stored in the learning target data storage section 22 as learning target data.

The training data extracting/correct answer data calculating unit 13 extracts, for each set of the medical record data stored in the learning target data storage section 22, vital data of predetermined examination items included in examination data in a first-year examination as a feature representing a health state of a user from the medical record data constituting the pair of the medical record data, and sets this examination data as training data. For example, HbA1c indicating a blood sugar level, a systolic blood pressure BP, and a body mass index (BMI) in the first-year examination are extracted.

In addition, the training data extracting/correct answer data calculating unit 13 calculates a risk score for co-occurrence or complications of a plurality of diseases based on the vital data of predetermined examination items and elapsed times until the plurality of diseases to be focused on occur. The vital data of the predetermined examination items is included in a feature representing a health state of the user, in other words, examination data in a first-year examination for each piece of medical record data constituting the set described above. At this time, the development risk score is calculated such that a user having a shorter elapsed time until development has a larger value than a user having a longer elapsed time until development. In addition, for medical record data under tracking and observation before occurrences of diseases, a development risk score is calculated using a length of a period until the tracking and observation become unexecutable as the elapsed time described above. Then, the training data extracting/correct answer data calculating unit 13 sets the calculated development risk score described above as correct answer data.

The prediction model learning unit 14 inputs training data extracted by the training data extracting/correct answer data calculating unit 13 described above to a learning machine and adjusts learning parameters of the learning machine such that an error is minimized between a score output from the learning machine at this time and the correct answer data calculated by the training data extracting/correct answer data calculating unit 13 described above. The learning machine, for example, is configured by a multi-layer neural network. Then, a prediction model in which learning parameters that have been finally acquired are reflected is stored in the prediction model storage section 23 as a learned prediction model. A specific example of a learning process performed by the prediction model learning unit 14 will be described below.

In a prediction phase, the evaluation data acquiring unit 15 performs a process of acquiring, as evaluation data, examination data of a user, who is a prediction target, for example, HbA1c, a systolic blood pressure, and a BMI, from the EMR server 2 or the access terminal 4 described above, for example, in response to a request from the access terminal 4. In addition, in this case, medical record data of the user may be acquired, and necessary examination data may be extracted from this medical record data as evaluation data.

The development risk score prediction processing unit 16 performs a process of inputting evaluation data acquired by the evaluation data acquiring unit 15 described above to the learned prediction model stored in the prediction model storage section 23 and transferring a development risk score output from the prediction model to the prediction data output unit 17.

The development risk score prediction processing unit 16 may store, in association with a user ID, a development risk score output from the learned prediction model in a prediction data storage section (not illustrated in the drawing) provided inside the storage unit 20.

The prediction data output unit 17 performs a process of generating prediction result notification data including the development risk score transferred from the development risk score prediction processing unit 16 described above and transmitting the generated prediction result notification data from the interface unit 30 to the access terminal 4 originating a request.

### Operational Examples

Next, an example of the operation of the state transition prediction device 1 configured as described above will be described.

### (1) Learning Phase

When a learning phase is configured, the state transition prediction device 1 performs the following prediction model learning process.

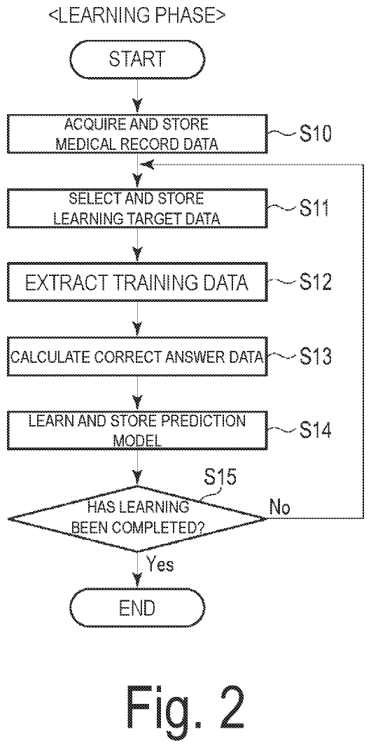

FIG. 2 is a flowchart illustrating an example of a processing sequence and processing details of a learning phase using the control unit 10 of the state transition prediction device 1.

(1-1) Acquisition of Medical Record Data

First, in step S10, the control unit 10 accesses the EMR server 2 through the interface unit 30 under control of the medical record data acquiring unit 11 and downloads individual medical record data relating to a plurality of users from the EMR server 2. Then, this medical record data is stored in the medical record data storage section 21 in association with a user ID. More medical record data may be acquired from the EHR server in addition to the EMR server.

When the medical record information described above is acquired, the medical record data acquiring unit 11 may acquire medical record data of all the users managed by the EMR server 2 and the like. The medical record data acquiring unit 11, for example, may search for and acquire only medical record data of users having a development history of a plurality of diseases designated in advance as learning targets, for example, diabetes and hypertension. This allows the storage capacity of the medical record data storage section 21 to be reduced and a processing load of a learning target data selecting process described below to be decreased. In addition, for example, in a case that user attributes such as a sex, an age group, a residential area, and an occupational category of a user are designated as learning targets, only medical record data of a user corresponding to such user attributes may be acquired.

(1-2) Selection of Learning Target Data

When the acquisition of medical record data of users is completed, next in step S11, the control unit 10 executes a process of selecting medical record data that is a learning target as below under control of the learning target data selecting unit 12.

In other words, first, the learning target data selecting unit 12 selects, from the medical record data storage section 21, medical record data relating to a user who has a development history of a plurality of diseases designated as learning targets in advance or a user for whom development of the plurality of diseases is under tracking and observation. For example, in a case that a co-occurrence or a complication of diabetes and hypertension is designated as a learning target, medical record data of a user who has a development history of diabetes and hypertension or a user for whom diabetes and hypertension are under tracking and observation is selected.

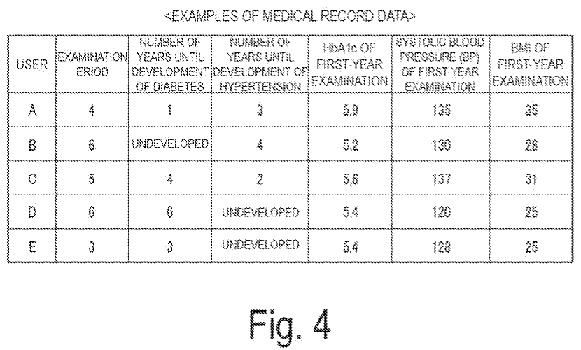

FIG. 4 illustrates an example of medical record data of users A to E, each having a development history of diabetes and hypertension or for whom the diseases are under tracking and observation, that has been selected in the process described above. This example indicates that each user name is associated with an examination period, an elapsed time until development of diabetes, an elapsed time until development of hypertension, HbA1c in a first-year examination, a systolic blood pressure (BP) in a first-year examination, and a BMI in a first-year examination.

Subsequently, the learning target data selecting unit 12 selects, from medical record data that has been selected as above, all the sets of medical record data, each set of medical record data having a common development pattern (for example, development order) of a plurality of diseases that are learning targets, for example, diabetes and hypertension, and different elapsed times until occurrence of such diseases.

Here, for a user for whom a disease has not yet developed symptoms, it is assumed that the disease has occurred at a point of time when tracking and observation become unexecutable, in other words, at any point of time after an examination period elapses, and an elapsed time until development is assumed to end at a similar point of time. Here, for example, any point of time is set to the day after a date of examination, or a date on which the next examination is scheduled after an examination period (a scheduled examination date of the next year or one year after the last examination date), or the day after a date of a last hospital visit after an examination period.

For example, in the example of medical record data illustrated in FIG. 4, hypertension has occurred after development of diabetes for a user A, and users D and E are selected as users having the same development pattern as the development pattern of the user A. Here, the users D and E are selected with the assumption that hypertension has developed in the seventh year after a medical checkup of the sixth year for the user D, and hypertension has occurred in the fourth year after a medical checkup of the third year for the user E. In other words, a set of the user A and the user D and a set of the user A and the user E are selected as learning targets.

In addition, diabetes has occurred after development of hypertension for the user C, and the user B is selected as a user having the same development pattern as the development pattern of the user C. Here, diabetes has occurred in the seventh year after a medical checkup of the sixth year for the user B, and the user B is selected. In other words, a set of the user B and the user C is set as a learning target. Then, the learning target data selecting unit 12 stores each of the selected sets of medical record data in the learning target data storage section 22.

In this way, although both a case of development and a case of non-development of a latter symptom out of two symptoms are configured as targets for selection, the selection may be restricted to users in whom all the symptoms have developed or users in whom a symptom has not developed. In addition, as sets of medical record data to be selected, all the sets of medical record data, each set of medical record data having a common development pattern (for example, development order) of a plurality of diseases that are learning targets, for example, diabetes and hypertension, different elapsed times until such diseases occur, and having an elapsed time until development of hypertension (or diabetes) and an elapsed time until development of diabetes (or hypertension) of a certain user that are shorter than an elapsed time until development of diabetes (or hypertension) and an elapsed time until development of diabetes (or hypertension) of the other user, may be selected from the medical record data. In addition, for a user including a symptom that has not developed, medical record data may be selected by applying similar conditions with the assumption that a corresponding disease has occurred at any point of time after tracking and observation become unexecutable and that an elapsed time until development ends at the similar time point.

(1-3) Extraction of Training Data and Calculation of Correct Answer Data

When the selection of learning target data described above is completed, under control of training data extracting/correct answer data calculating unit 13, the control unit 10, first, in Step S12, reads learning target data from the learning target data storage section 22. Next, the training data extracting/correct answer data calculating unit 13 extracts, from such learning target data, HbA1c, a systolic blood pressure, and a BMI, which are examination data of a first-year examination, as feature indicating a health state of the user. The feature indicating a health state of a user may be any other values that can be quantitatively represented by items capable of contributing to the calculation of a score in a specimen examination, a physiological examination, or the like.

As a result, for example, HbA1c “5.2”, systolic blood pressure “130”, and BMI “28” are extracted from the medical record data of the user B, and HbA1c “5.6”, systolic blood pressure “137”, and BMI “31” are extracted from the medical record data of the user C. Then, this extracted examination data of the users is used as training data.

Subsequently, in Step S13, the training data extracting/correct answer data calculating unit 13 calculates a development risk score of a complication for each set of medical record data stored in the learning target data storage section 22 described above as learning target data based on HbA1c, the systolic blood pressure, and the BMI, which are examination data of a first-year examination and the elapsed time until diabetes has occurred and the elapsed time until the hypertension has occurred for each piece of medical record data constituting a set.

Here, at this time, the development risk score is calculated such that the score of a user having a short elapsed time until development is higher than the score of a user having a long elapsed time until development. For a user under tracking and observation before the occurrence of a disease, the score is calculated using the length of a time until tracking and observation become unexecutable as the elapsed time described above. Then, the training data extracting/correct answer data calculating unit 13 sets the development risk score calculated as above as correct answer data.

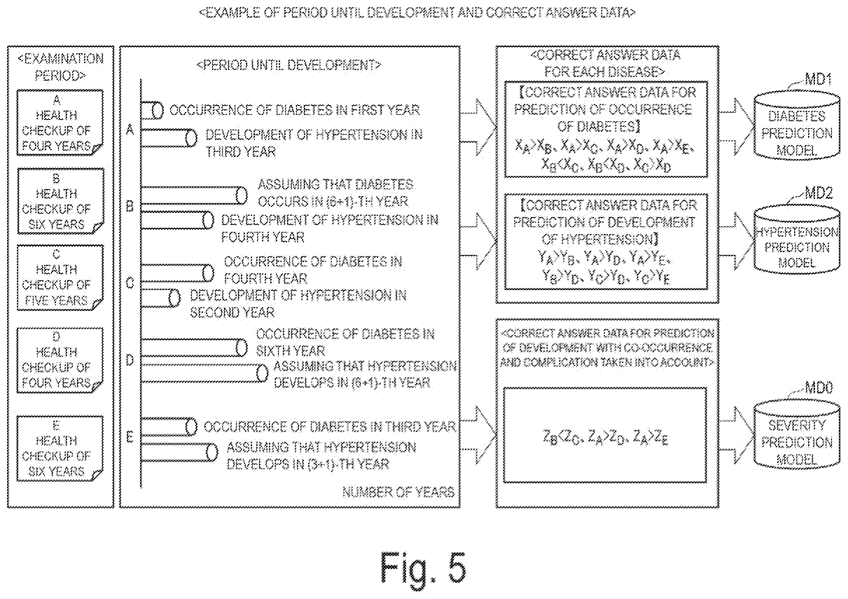

FIG. 5 represents periods until development of diabetes and hypertension of the users A to E illustrated in FIG. 4 using bar graphs and illustrates an example of correct answer data of development risk scores with co-occurrence or complications additionally taken into account. In this example, a set of the user B and the user C, a set of the user A and the user D, and a set of the user A and the user E are selected as learning targets by the learning target data selecting unit 12 described above, and thus a score is calculated based on medical record data of each of the sets.

For example, for the set of the user B and the user C, the user C has a shorter elapsed time until development than the user B, and thus the score Z_{C} of the user C is calculated to be higher than the score Z_{B} of the user B, in other words, Z_{B}<Z_{C}. In addition, for the set of the user A and the user D, the user A has an shorter elapsed time until development than the user D, and thus, the score Z_{A} of the user A is calculated to be higher than the score Z_{D} of the user D, in other words, Z_{A}>Z_{D}. Similarly, for the set of the user A and the user E, diabetes occurred in the third year, and hypertension has not developed in the period of health checkup of three years for the user E, and thus, the score Z_{A} of the user A is calculated to be higher than the score Z_{E} of the user E, in other words, Z_{A}>Z_{E}.

(1-4) Learning of Prediction Model

Next, under control of the prediction model learning unit 14, the control unit 10 executes a process of learning a prediction model in Step S14.

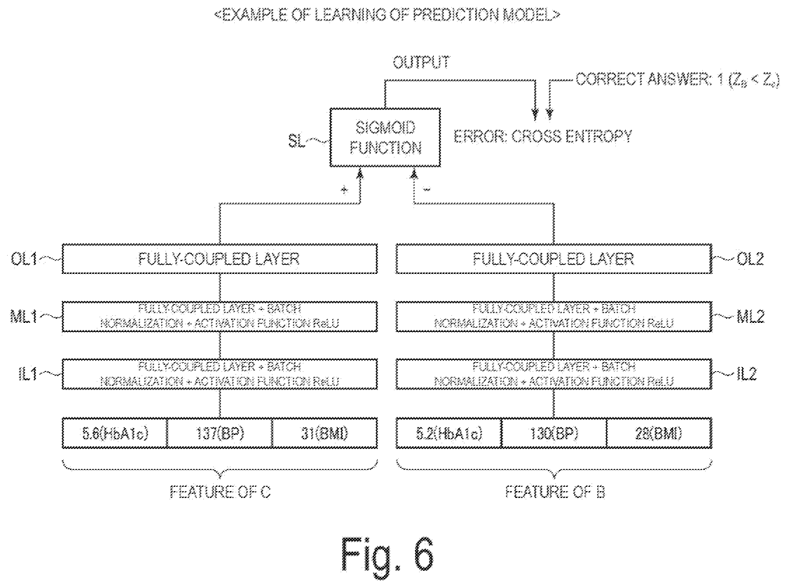

FIG. 6 illustrates an example of the configuration of a learning machine used for learning a prediction model, and, for example, a multi-layer neural network is used as the learning machine. The multi-layer neural network, for example, is configured by three layers including input layers IL1 and IL2, intermediate layers ML1 and ML2, and output layers OL1 and OL2. Among these, each of the input layers IL1 and IL2 and the intermediate layers ML1 and ML2 is configured by a fully-coupled layer, a batch normalization, and an activation function ReLU, and each of the output layers OL1 and OL2 is configured by a fully-coupled layer.

The prediction model learning unit 14 inputs to the input layers IL1 and IL2 examination data of a first-year examination extracted from each piece of medical record data of users constituting a set as training data by using the training data extracting/correct answer data calculating unit 13. For example, for the set of the user B and the user C as an example, HbA1c “5.2”, systolic blood pressure “130” and BMI “28” that are examination data of a first-year examination of the user B and HbA1c “5.6”, systolic blood pressure “137” and BMI “31” that are examination data of a first-year examination of the user C are input to the input layers IL1 and IL2 of two systems of the learning machine.

The prediction model learning unit 14 inputs to a calculation unit SL of a Sigmoid function a difference between a score corresponding to the examination data of the first-year examination of the user B and a score corresponding to the examination data of the first-year examination of the user C, which have been output from the output layers OL1 and OL2 of the learning machine. Then, a cross entropy between an output value thereof and a correct answer value “1” that is acquired from a relationship “Z_{B}<Z_{C}” between correct answer data of the user B and correct answer data of the user C calculated by the training data extracting/correct answer data calculating unit 13 described above is calculated and is set as an error. Then, the error is minimized using an optimization method of Adam.

In other words, a three-dimensional vector of examination data is input to the input layers IL1 and IL2 of the learning machine, and scores formed from one-dimensional vectors are output from the output layers OL1 and OL2. In other words, the unit size of the input layer of the learning machine is “3”, and the unit size of the output layer is “1”. The unit size of the intermediate layer is “64”. The parameters are not limited thereto, and the unit size may be changed appropriately in accordance with the number of items used for calculating a score and the relationship among the items.

For sets of all the learning target data stored in the learning target data storage section 22, similar to the above-described case of the user B and the user C, the prediction model learning unit 14 inputs examination data of a first-year examination to the learning machine as training data. In addition, the prediction model learning unit 14 calculates an error of a cross entropy between a Sigmoid function value of a difference between outputs of the learning machine and a correct value acquired from the relationship of correct answer data and performs an optimization process of minimizing this error. Then, when the completion of the learning process using all the learning target data is detected in Step S15, a prediction model in which the learning parameters at the time point have been reflected is stored in the prediction model storage section 23 as a learned prediction model, and the process of learning a prediction model ends.

In FIG. 5, a case that individual correct answer data is calculated for diabetes and hypertension is also illustrated for a reference. In other words, when a risk score for diabetes calculated from the first-year examination data of each user is denoted by X, and a risk score for hypertension is denoted by Y, correct answer data satisfying the magnitude relationships X_{A}>X_{B}, X_{A}>X_{C}, X_{A}>X_{D}, X_{A}>X_{E}, X_{B}<X_{C}, X_{B}<X_{D}, X_{C}>X_{D} and Y_{A}>Y_{B} for diabetes and Y_{A}>Y_{D}, Y_{A}>Y_{E}, Y_{B}>Y_{D}, Y_{C}>Y_{D}, and Y_{C}>Y_{E} for hypertension is set. Then, when the learning machine is caused to perform learning using such correct answer data, a prediction model for diabetes and a prediction model for hypertension can be generated. Thus, by using such prediction models, a development risk of only diabetes and a development risk of only hypertension can be predicted as well.

(2) Prediction Phase

When a prediction phase is set, the state transition prediction device 1 performs a process of predicting, for a user, a development risk of a co-occurrence or a complication of a plurality of diseases in the future as below.

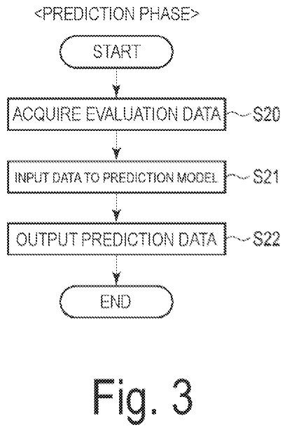

FIG. 3 is a flowchart illustrating an example of a procedure and processing details of a prediction process performed by the control unit 10 of the state transition prediction device 1.

(2-1) Acquisition of Evaluation Data

When examination data of a user who is a prediction target is input to the state transition prediction device 1, the control unit 10 imports the examination data described above through the interface unit 30 as evaluation data under control of the evaluation data acquiring unit 15 in step S20. Examples of the examination data to be input include HbA1c, a systolic blood pressure, and a BMI, which are vital data representing feature of the current health state of the user who is a prediction target. The process of inputting the examination data of the user who is the prediction target described above, for example, is performed by a terminal of a medical-related person such as a doctor, a user terminal, or a terminal of an insurance company.

(2-2) Prediction of Development Risk Score

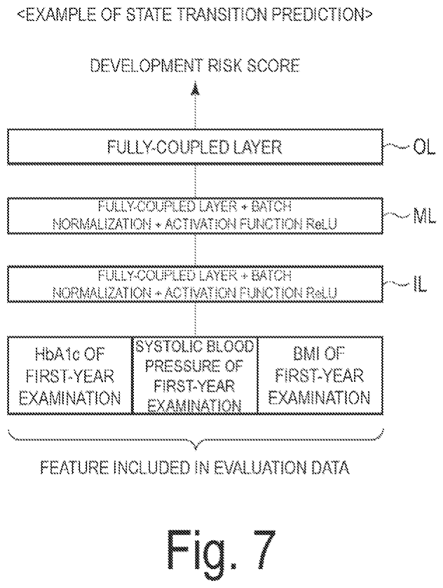

When the importing of the evaluation data described above is completed, the control unit 10 of the state transition prediction device 1 executes a process of predicting a development risk score as below under control of the development risk score prediction processing unit 16. FIG. 7 is a diagram illustrating processing details thereof.

In other words, the development risk score prediction processing unit 16 reads a learned prediction model stored in the prediction model storage section 23. Then, in step S21, the evaluation data, for example, HbA1c, the systolic blood pressure, and the BMI acquired as above are input to the input layer IL of the learned prediction model described above. Then, in the learned prediction model, a prediction score is calculated by the input layer TL and the intermediate layer ML using a three-dimensional vector constituted by HbA1c, the systolic blood pressure, and the BMI as an input, and a development risk score represented by a one-dimensional vector is output from the output layer OL.

(2-3) Output of Prediction Data

Under control of the prediction data output unit 17, the control unit 10 generates prediction result notification data including a development risk score output from the learned prediction model in step S22. In the prediction result notification data, although the development risk score may be included without change, a degree of a development risk acquired by determining the development risk score using a threshold may be included, and an advice message according to the degree of the development risk or the like may be included.

The prediction data output unit 17 transmits the prediction result notification data described above from the interface unit 30 to a terminal of a medical-related person, a user terminal, or a terminal of an insurance company that originates a request. As a method of the transmission, the prediction result notification data may be transmitted in a form that can be read using a browser of a terminal or may be transmitted in a form of being attached to an electronic mail.

Effect

As described above, according to one embodiment of the present invention, in the learning phase, from medical record data having a development history of a plurality of diseases having a likelihood of co-occurrence or occurrence of complications or medical record data under tracking and observation of development of the diseases, a set of medical record data having a common development order of diseases to be focused on, and different elapsed times until occurrences of the diseases is selected. Then, for each set of medical record data, examination data of the first-year examination is extracted from each medical record data constituting the set as a feature representing the health state of the user, and the examination data is set as the training data. In addition, a risk score for a co-occurrence or an occurrence of a complication of the plurality of diseases is calculated based on the examination data of the first-year examination and elapsed times until occurrences of the plurality of diseases and is set as correct answer data. At this time, the development risk score is calculated such that a user having a short elapsed time until development has a larger value than a user having a long elapsed time until development. Then, the training data described above is input to the learning machine, and the learning machine is caused to learn such that the output becomes the correct answer data described above, whereby a learned prediction model is generated.

Thus, in a case that there is a likelihood of a co-occurrence or an occurrence of a complication of a plurality of diseases, a prediction model in which a development pattern of the plurality of diseases, in other words, a development order and elapsed times until the occurrences are taken into account can be generated.

According to embodiment of the present invention, in a prediction phase, examination data of a user who is a prediction target is input into the learned prediction model, and prediction result data including a development risk score output from the prediction model is output. For this reason, based on the current examination data of a user, the development risk of a co-occurrence or an occurrence of a complication of a plurality of diseases in the future can be predicted for the user.

### Other Embodiments

The embodiment described above may be modified as follows. In other words, for example, one or more sets of the following three sets of users are selected as learning target data out of acquired use data. A first set of users is a set of users having occurrence of one disease to be focused on and different elapsed times until development. A second set of users is a set of users having no occurrence of a disease and different elapsed times that are extended until a point of time after tracking becomes unexecutable. A third set of users is a set of users including a user having occurrence of a disease and a user having no occurrence of a disease, and an elapsed time until development of the user having the occurrence of the disease is different from an elapsed time, of the user having no occurrence of the disease, that is extended until a point of time after the tracking becomes unexecutable. Next, in the extraction of training data and the calculation of correct answer data, a model may be learned such that an error between a score output by the prediction model based on feature of a non-developed state and a risk score calculated based on an elapsed time until a disease occurs for the user or an elapsed time extended until a point of time after the tracking becomes unexecutable is minimized for development risk scores defined such that the score becomes higher for a shorter elapsed time until an occurrence of one disease to be focused on.

By assuming that a disease occurs for a user having no development of a symptom after a point of time when the tracking and observation become unexecutable and setting the user as a learning target, there is an effect of improvement of accuracy according to an increase in the number of targets.

In the embodiment described above, the state transition prediction device having both functions of a functional unit for learning a prediction model and a functional unit for predicting a development risk score for predicting a development risk score using the learned prediction model has been described as an example. However, according to the present invention, a learning device including only a functional unit for learning a prediction model and a prediction device including only a functional unit for predicting a development risk score may be configured as separate devices.

In addition, in the embodiment described above, a case that a development risk of a co-occurrence or a complication of a plurality of diseases in the future is predicted based on examination data indicating current health states of users in the field of medical health has been described as an example. However, the present invention is not limited thereto and can be applied to other fields as long as state transitions can be observed. For example, the present invention can be applied to transportation apparatuses such as a vehicle, an aircraft, a ship, and the like, a manufacturing apparatus, a power equipment, an office device, a medical device, a power device, and the like such that an object is targeted that has a plurality of parts that can possibly malfunction and the likelihood of malfunction based on a state of a device at a point of time is represented using a uniform score regardless of the order of the malfunctions.

In short, the present invention is not limited to the above-described embodiment as it is, and can be embodied with the components modified without departing from the scope of the disclosure when implemented. Furthermore, various inventions can be formed by appropriate combinations of a plurality of components disclosed in the above-described embodiment. For example, several components may be deleted from all of the components illustrated in the embodiment. Furthermore, components of different embodiments may be appropriately combined with each other.

### REFERENCE SIGNS LIST

1 State transition prediction device
2 EMR server
3 Network
4 Access terminal
10 Control unit
11 Medical record data acquiring unit
12 Learning target data selecting unit
13 Training data extracting/correct answer data calculating unit
14 Prediction model learning unit
15 Evaluation data acquiring unit
16 Development risk score prediction processing unit
17 Prediction data output unit
20 Storage unit
21 Medical record data storage section
22 Learning target data storage section
23 Prediction model storage section
30 Interface unit

1. A state transition prediction device comprising:
a processor; and
a storage medium having computer program instructions stored thereon, when executed by the processor, perform to:
acquire feature data including a feature relating to a first state, an elapsed time until the first state transitions to a second state, and an elapsed time until the first state transitions to a third state in a case that a health state of a user transitions from the first state to the second state due to an occurrence of a first symptom and transitions from the second state to the third state due to an occurrence of a second symptom;
select, from the acquired feature data, first feature data and second feature data, in which the first symptom of the first feature data is identical to the first symptom of the second feature data, the second symptom of the first feature data is identical to the second symptom of the second feature data, and elapsed times of state transitions are different from each other; and
generate a prediction model by setting the feature relating to the first state included in each of the first feature data and the second feature data as training data and causing a learning machine to learn prediction scores which are respectively calculated based on the features and reflect the elapsed times respectively included in the first feature data and the second feature data as correct answer data.

2. The state transition prediction device according to claim 1, wherein the computer program instructions further perform to acquire the feature relating to the first state indicating the health state of the user who is a prediction target, input the feature to the prediction model as evaluation data, and output a prediction score output from the prediction model in accordance with the input as information representing a prediction result of a future state transition of the health state of the user who is the prediction target.

3. The state transition prediction device according to claim 1, wherein to the feature data includes a length of a time until tracking of the state transition becomes unexecutable as the elapsed time in a case that the first state has not transitioned to the second or third state.

4. A state transition prediction method executed by a state transition prediction device including a computer, the state transition prediction method comprising:
acquiring feature data including a feature relating to a first state, an elapsed time until the first state transitions to a second state, and an elapsed time until the first state transitions to a third state in a case that a health state of a user transitions from the first state to the second state due to an occurrence of a first symptom and transitions from the second state to the third state due to an occurrence of a second symptom; selecting, from the acquired feature data, first feature data and second feature data, in which the first symptom of the first feature data is identical to the first symptom of the second feature data, the second symptom of the first feature data is identical to the second symptom of the second feature data, and elapsed times of state transitions are different from each other; and
generating a prediction model by setting the feature relating to the first state included in each of the first feature data and the second feature data as training data and causing a learning machine to learn prediction scores which are calculated based on the features and reflect the elapsed times respectively included in the first feature data and the second feature data as correct answer data.

5. The state transition prediction method according to claim 4, further comprising acquiring the feature relating to the first state indicating the health state of the user who is a prediction target, inputting the feature to the prediction model as evaluation data, and outputting a prediction score output from the prediction model in accordance with the input as information representing a prediction result of a future state transition of the health state of the user who is the prediction target.

6.-10. (canceled)
